(19) United States
(12) Patent Application Publication
(10) Pub. No.: US 2021/0290580 A1
(43) Pub. Date: Sep. 23, 2021

(54) New use of carbamate ß phenylethanolamine analogues for enhancing intracellular clearance of LDL cholesterol and for combining therapy with statins to enhance the efficacy and reduce adverse effects

(71) Applicant: Wen TAN, Guangdong (CN)

(72) Inventor: Wen TAN, Guangdong (CN)

(21) Appl. No.: 17/264,798

(22) PCT Filed: Jul. 25, 2019
(86) PCT No.: PCT/US2019/043328

§ 371 Date: Jan. 30, 2021

Related U.S. Application Data
(60) Provisional application No. 62/712,384, filed on Jul. 31, 2018.

(51) Int. Cl. A61K 31/221; A61K 31/216; A61K 45/06; A61K 31/40; A61P 3/06

(52) U.S. Cl. CPC A61K 31/221; A61K 31/216; A61P 3/06; A61K 31/40; A61K 45/06

(57) ABSTRACT

The invention related to the use of carbamate-β-phenylethanolamine analogues for up-regulating of LDL receptors, facilitating uptake of extracellular LDL cholesterol and reducing intercellular total cholesterol. It also related to use of carbamate-β-phenylethanolamine analogues and statins or other lipid lowering agents as combined therapies for synergic effects in lowering LDL cholesterol as well as for reducing adverse effects.

### FIELD OF INVENTION

This invention related to the use of carbamate-β-phenylethanolamine analogues including R-bambuterol for up-regulating LDL receptors and for facilitating the uptake of LDL cholesterol by hepatic and other cells. This invention also related to the use of carbamate-β-phenylethanolamine analogues including R-bambuterol for enhancing the turnover or clearance of intercellular LDL cholesterol, therefore facilitating the clearance of LDL cholesterols from blood. Lowering LDL cholesterol is the most important goal in the treatments of hyperlipidemia. A causative relationship between LDL cholesterol and cardiovascular risks has been well established [Michael G. Silverman, et al. Association Between Lowering LDL-C and Cardiovascular Risk Reduction. JAMA Volume 316: 12, Sep. 27, 2016]. However, it still remains controversy about the relationship between cardiovascular risk and the levels of blood total cholesterol or triglyceride [Meera Senthilingam, Statins or not? New study aims to help doctors and patients decide, CNN, Sep. 8, 2016; Robert DuBroff, Michel de Lorgeril, Cholesterol confusion and statin controversy, World J Cardiol 2015 Jul. 26; 7(7): 404-409].

LDL receptor and LDL cholesterol is the most important therapeutic targets for clinic management of hyperlipidemia. The main target or mechanism of commonly use statins is to block the synthesis of total cholesterol. The disadvantages for statins are the adverse effects. The major adverse effects were muscular myalgia, diabetes and liver damage, neuronal and etc. These off targets effects of statins besides lowering blood cholesterol were attributed to a non-selective inhibition of cholesterol synthesis in our tissues by statins, since cholesterol is also the building block for various lipids and hormones which play important roles in physiological functions. It is desirable to provide medicine which can precisely target the intracellular LDL cholesterol and has fewer adverse effects. Few drugs are available for the purpose. This invention disclosed a new class of medicine for the unmet medical need, which specifically acts on LDL receptors and enhance the clearance of intracellular LDL cholesterol, in practically the liver cells.

Statins has been used for more than 20 years and now count about 90% of the total market for treatment hyperlipidemia. Statin is still the most popular used drugs although they have serious adverse effects. In addition, statin therapies are ineffective for some of 30% patients. This invention also related to a used of carbamate-β-phenylethanolamine analogues including R-bambuterol in combination with statins and other lipid lowering agents in treatment of hyperlipidemia in order to achieve enhanced and synergistic therapeutic efficacy in lowering LDL cholesterol. This invention also related to a used of carbamate-β-phenylethanolamine analogues in combination with statins and other lipid lowering agents in order to reduce the toxicities and adverse effects induced by statins and other lipid lowering agents.

### BACKGROUND OF INVENTION

Dyslipidemia is a wide spread decease. It may involve increased blood triglycerides, total cholesterol, high density lipoprotein combined cholesterol “HDL” and low density lipoprotein (LDL) combined cholesterol. The major clinic complications for dyslipidemia are the damage of major arteries due to a deposition of cholesterol and inflammatory responses, which results in arteriosclerosis and plaques formation. Arteriosclerosis of resistance arterioles could lead to hypertension. While a damaged coronary or cerebral arterials could cause tissue ischemia and furthermore, a fall of plaques from vascular wall could immediately form a blockage resulting in heart attach or brain stroke. It is general understands in medical community that hyperlipidemia will increase the risk of cardiovascular diseases. Recently there are controversy about whether there is a causative relationship between high blood lipid and cardiovascular incidences, since the meta-analysis of clinic trials shows that high blood cholesterol does not results higher incidences of cardiovascular diseases, which significant number of cardiovascular diseases were found in patients with a normal or even lower blood lipid or cholesterol. Nonetheless, it is well accepted and confirmed by major clinic trials that there was a clear causative relationship between blood LDL cholesterol and the risk of cardiovascular diseases. The lower of the level of blood LDL cholesterol often related to the more beneficial effects on the cardiovascular outcomes in the patients. Therefore, a major goal for current clinic management of dyslipidemia is still to lower the level of blood LDL cholesterol.

Lipophilic Statins has been the major medicine for lipid lowering, particularly for lowering LDL cholesterol in past decades. The main mechanism of action for statin is to block the synthesis of cholesterol by competitively inhibiting a key-enzyme, HMG-CoA reductase. However, There are about more than 30% of patients who are not response to the treatment of statin. In addition, HMG-CoA reductase is also a key-enzyme involves in the synthesis of other steroid compounds in the body such as corticosteroids, sex hormones etc. Therefore, long term of use statins causes serious adverse effects such as myalgia and risk of diabetes, hepatotoxicity and even cardiac problems by the recent report [Francois Mach et al., Adverse effects of statin therapy: perception vs. the evidence—focus on glucose homeostasis, cognitive, renal and hepatic function, hemorrhagic stroke and cataract, European Heart Journal, Volume 39, Issue 27, 14 Jul. 2018, Pages 2526; L P Cahalin et al., Opposite effect of statins on pulmonary function and exercise tolerance in diastolic versus systolic heart failure Chest. 136 (4) (2009)].

And there are about 30% patients who are resistant to statins therapy. It is desirable to provide medicine that can more precisely target the LDL cholesterol and has less adverse effects. Few drugs are available for the purpose. This invention disclosed a new class of medicine for the unmet medical need, which specifically act on LDL receptors and lowering intracellular LDL cholesterol.

Ezetimibe represent another class of lipid lowering medicine. Ezetimibe is an inhibitor of intestinal cholesterol absorption by inhibiting the Niemann-Pick C1-like 1 protein (NPC1L1), which is involved in cholesterol transport in the liver as well as in the intestine. It therefore often be used in combination with inhibitor of cholesterol synthesis statin, in order to achieve better blood lipid lowering effects.

An recently success in the treatment of dyslipidemia is the marketing of newly developed drug PCSK9 antibody which is given intravenously. PCSK9 antibody can significantly reduce blood LDL cholesterol. The mechanism of action of PCSK9 is different comparing to statins, It blocks a specific protein in the blood called PCSK9. PCSK9 has been found to act as protease which can break down the LDL receptors in the cytosol, and therefore reduces the rate of turn-over and the amount of LDL which return back to the membrane. The reduction of LDL receptors decreases the transport and metabolite of blood LDL cholesterol, and results an increase of blood LDL cholesterol. The elevated LDL cholesterol in patients will be significant reduced by block the PCSK9 protein using PCSK9 antibody [Fiorella Devito et. at. Focus on alirocumab: A PCSK9 antibody to treat hypercholesterolemia. Pharmacological Research. Vol. 120, December (2015)].

However, the compliance of patients to the intravenous injection formulation of PCSK9 is major drawback for its clinic application since dyslipidemia is chronic and is not a immediate or directly life threatening diseases. In addition, there are a numbers of clinic trials reveals some serious adverse affects such cognitive disorders in patients received PKSC9 antibody treatments [Swiger K J and Martin S S. PCSK9 inhibitors and neurocognitive adverse events: exploring the FDA directive and a proposal for N-of-1 trials. Drug Saf. June; 38(6):519-26, (2015)].

R-bambuterol has lipid-lowering effects in mice and human subjects [Wen Tan, R-Bambuterol, its preparation and therapeutic uses, EP20020807678.] R-bambuterol lowers the total cholesterol and LDL cholesterol in blood when given orally [Ye et al. The Lipid-lowering Effects of R-bambuterol in Healthy Chinese Volunteers: A Randomized Phase I Clinical Study, EBioMedicine 2 (2015) 356].

However, blood cholesterol may be affected by many different ways. It could be affected by intestinal absorption, synthesis, bile acid sequestrants, cholesterol transport between blood and cells, and cholesterol up-take by hepatic or other organs, the activities or function of LDL receptors etc. The mechanism or targets of action for R-bambuterol is not clear so far. The roles of R-bambuterol on LDL receptors as well as its roles on intracellular LDL cholesterol turnover remain unknown.

It may be related to an inhibition of the activity of Butyric cholinesterase (BuChE) since R-bambuterol is one of the most potent inhibitor of BuChE. Highly expression or enhanced activity of BuChE were related to hyperlipidemia and obesity [Kutty, K M et, al. Serum pseudocholinesterase: high density lipoprotein cholesterol ratio as an index of risk for cardiovascular disease, Clinica Chimica Acta 115(1):55-61, February (1981); Calderon-Margalit et al.: BuChE, Cardiovascular Risk Factors, and Mortality, General Clinical Chemistry 52:5000 (2006)]. However, there is also a controversy. A causative relationship between BuChE and LDL or cholesterol has not been established. It is reported that in BuChE knockout mice, the body weight was increased than wild type mice when feed with high fat diets [Chen Y P et al., Butyric cholinesterase deficiency promotes adipose tissue growth and hepatic lipid accumulation in male mice on high fat diet. Endocrinology 157: 3086-3059, (2016)].

R-bambuterol is a pro-drug of terbutaline. The effects on LDL receptor and LDL cholesterol disclosed in this invention were not related to its parent drug of terbutaline according to our investigation. Furthermore, the relationship between BuChE and LDL receptor as well as BuChE inhibitors and LDL receptor has never been studied. In the prior art, it remains unknown that what are the targets responsible for lowering blood lipid and LDL cholesterol by R-bambuterol. Whether R-bambuterol act on LDL receptors or on the clearance of intracellular cholesterol or both have not been studied [Wen Tan, R-Bambuterol, its preparation and therapeutic uses, EP20020807678].

In addition, R-bambuterol is a pro-drug of terbutaline which is a β2 agonist. It believes that β2 agonist regulates a number of pathways involved in lipid and may lower the cholesterol and LDL-C. β2 agonist like terbutaline may stimulate sterol regulatory element-binding proteins (SREBPs) which regulating the biosynthesis of cholesterol, fatty acids and triglycerides. It believes that the lipid lowering effects of R-bambuterol may attribute to terbutaline the parent drug. Therefore, the fact that R-bambuterol lowering the lipid is a demonstration of the roles of beta-2 agonists in lipid metabolism, which may provide another class of agents that address an unmet clinical need in patients with dyslipidemia [Michael H. Davidson, Beta-2 Agonism: A Potential Therapeutic Target for Dyslipidemia. EBioMedicine 2 (2015) 284].

The unmet medical need for dyslipidemia is to find a medicine which can act specifically on the receptor of LDL cholesterol and on the intracellular clearance of LDL cholesterol with reduce adverse effects. In addition, to find a medicine which may reduce the toxicity and enhance the efficacy of statins which have become the major anti-lipid drugs today.

### Detail of Invention

In an embodiment, the invention disclosed a new use of R-bambuterol. This invention revealed that R-bambuterol can facilitate the endocytosis or transport of LDL cholesterol as well as oxidized LDL (ox-LDL) from extracellular space into hepatic cells using a fluorescence labeled LDL cholesterol. In addition, there is also up-regulation of expression of LDL receptors in the cell membrane as well as increase binding of LDL cholesterol to the receptors after treated with R-bambuterol. These were demonstrated using both fluorescence labeled LDL-C and LDL receptor specific antibody. As results, R-bambuterol facilitates the transport of LDL-C and ox-LDL-C into cells and eliminates the LDL and ox-LDL cholesterol from extracellular space or blood, therefore, lowering the level of LDL and ox-LDL cholesterol.

This invention disclosed that the targets and the mechanisms of R-bambuterol in lowering LDL cholesterol are distinct from statins that is mainly by inhibiting the intracellular synthesis of cholesterol. The therapeutic target of R-bambuterol disclosed in this invention is also differ than PCSK9 antibody which slow down the intercellular degradation of LDL receptor by inactivating the PCSK9 enzyme. This finding of new use of R-bambuterol in this invention have not been reported in prior art and can not be anticipated by a skill person in art. It is therefore should be considered as novel and involves a inventive step.

In one embodiment, invention also provides that there were significant reductions of hepatic intercellular LDL cholesterol when treated with R-bambuterol in a dose-dependent fashion. In the meantime, there was an inhibition of the activity of butyric cholinesterase.

It is known that oral administration of R-bambuterol can reduce the blood lipid and LDL in prior art by W Tan & J Chen [US patent. 2002]. However, whether it involves in absorption, elimination or other targets or mechanisms remains unknown. This enhanced clearance or turnover of intercellular LDL cholesterol by R-bambuterol, which may be either via increased metabolite, increased bile acid secretion in the case of hepatic cell, or via reduced synthesis of cholesterol. These effects on LDL receptor disclosed in this invention have nerve been reported in prior art. Lipid disorder or hyperlipidemia is symptoms involve difference mechanisms or disease targets. It is desirable to treatment patients with medicine, which has clear and specific physiological targets. This invention provides a new used of class compounds for treatment of increased LDL cholesterol to the patients in need.

Statins are the first-line and widely prescript medicine. It is desirable to combine R-bambuterol with statin to achieve better effects. This invention disclosed that there is a significant synergistic effect when both R-bambuterol and statins are used in combining.

In one embodiment this invention reveals that neither R-bambuterol nor statins showed inhibitory effects on the reduction or synthesis of intracellular cholesterols at a relatively lower dose. However, when both R-bambuterol and used in combination at the same doses above, there is a significant reduction of intracellular cholesterols. Furthermore, in comparison of the up-taking or transport of LDL from extracellular space into cells, the combination of R-bambuterol and statin have significantly more effects in comparison of either R-bambuterol or statin alone. This reduction or clearance of intracellular cholesterol can be attributed to decreased cholesterol synthesis and increased metabolites to other compounds or secreted by hepatic cell in the form of bile acid. This synergistic effect has never been reported in prior art. It can not be anticipated by a person in art.

Furthermore, it is known that statin may induce hepatic toxicity and other adverse effects [Atorvastatin associated liver disease, Clarke A T and P R. Mills, Digestive and liver disease, 38: 772. (2006)]. Aminotransferase levels, an indicator for liver damaging were >3 times upper limit of normal (ULN) are seen in up to 2% of the patients exposed to statins [Chalasani N, et al, Patients with elevated liver enzymes are not at higher risk for statin hepatotoxicity. Gastroenterology. 126(5):1287-1292. (2004)]. Due to the adverse effects, statins are intolerable for certain patients which have to terminate the therapy or a re-adjustment of dosages which renders the statin therapy less effective.

In one aspect, the invention reveals that Atorvastatin treatment can significantly inhibit intercellular cholesterol synthesis by the cells, but at the same time it is also toxic to the cells and reduces the viability of cells with increased doses. On the other hand, Bambuterol has no toxicity toward the cells at the dose of 4× more as statin. In the meantime it showed a significant reduction of intercellular cholesterol. R-bambuterol is superior than statins in this regard.

In one embodiment, treated the cells with a combination of Atorvastatin and R-bambuterol. We found by surprise that the toxic effects of Atorvastatin were greatly reduced. Atorvastatin inhibit significantly the cell viability at doses of 10 μM or 20 μM. However, there were no inhibition in cell viability was observed when treated with the same dose of Atorvastatin in combination with 20 μM of R-bambuterol. Therefore, R-bambuterol can protect cells against the toxicity induced by Atorvastatin and render the cells more tolerance to the treatment of Atorvastatin and other statins. These have never been reported by prior art and it should be considered as novel and inventive.

In one embodiment, we disclosed that in hyperlipidemia rabbits induced by high fat and high cholesterol diet, Atorvastatin in combining with R-bambuterol showed more lipid lowering effects than Atorvastatin alone. In addition, the toxicity effects by pathological examining were significant in Atorvastatin alone, but no obvious damage were observed in groups combination of statin and R-bambuterol.

In another embodiment we disclosed similar protective effects of R-bambuterol were also found in skeleton or smooth muscles cells and cardiomyocytes.

In one hand, as mentioned before, R-bambuterol increases the LDL receptors expression and facilitates the internalization of LDL. In the other hand, R-bambuterol reduces the intracellular cholesterol and enhances the clearance of intracellular LDL cholesterol. These roles of R-bambuterol in regulating LDL receptors and enhancing intracellular LDL cholesterol clearance are novel and can not be anticipated by a person in art.

R-bambuterol is a pro-drug of terbutaline. It was believed that the lipid lowering effects of R-bambuterol is due to the effects of its parent [Michael H. Davidson, Beta-2 Agonism: A Potential Therapeutic Target for Dyslipidemia, Ebiomedicine 2015]. In one embodiment, this invention provides that neither of the reduction of LDL nor the cellular protection against statins were related to terbutaline. Therefore, this invention overcoming a widely-accepted prejudice, and disclosed the lipid lowering effect of R-bambuterol is not related to its parent drug terbutaline. It is not obvious from prior arts.

R-bambuterol is a pro-drug of terbutaline, R-bambuterol has been demonstrated to be a potent BuChE inhibitor, while its parent drug terbutaline did not. Furthermore, the protective effect of R-bambuterol is related with the inhibition of BuChE. Only R-enantiomer of bambuterol was found to be most effective in lipid lowering, while the S-enantiomer is less or no effects. It indicates that BuChE is chiral selective.

In one embodiment in the invention provides that the similar protective effects R-bambuterol against the toxicities of Simvastatin was seen when both drugs were used in combination. R-bambuterol can be protective against the toxicities induced by other drugs in the statin class, typically commercial available statins such as lovastatin, paravastatin, rusovastatin cerivastatin, fluvastatin, mevastatin, pitavastatin, pravastin and etc.

In another embodiment in the invention reveals that R-mono carbonamide—bambuterol or Ethylating bambuterol also have similar effects on LDL receptors and intracellular LDL cholesterol clearances. They have synergistic effects when used in combining with Statins. In addition, they have similar protective effective against the toxicities induced by either Atorvastatin or Simivastatin and other statins as did by R-bambuterol when each one of the compounds was used separately in combination with one of those statins.

This invention disclosed that a group of (±)-carbamate-β-phenylethanolamine analogues described in formula I and their active enantiomers as well as their pharmaceutically suitable salts have the similar inhibitory functions in butyric cholinesterase and have the similar effects as R-bambuterol in up-regulating LDL receptors and in enhancing the clearance of intracellular LDL cholesterol via synthesis or metabolize as well as in protecting against the adverse effects or toxicities induced by statins and other lipid lowering agents.

Furthermore, this invention overcomes a widely-accepted prejudice that R-bambuterol and its analogues play its lipid lowering roles via its parent drug terbutaline.

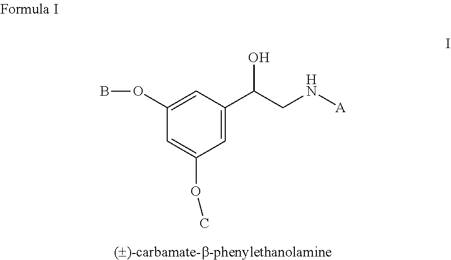

Wherein

A is selected from substituted or un-substituted alkyl, substituted or un-substituted alkenyl, substituted or unsubstituted aryl, substituted or un-substituted cycloalkyl;
B is selected from hydrogen or —CO—N(W)(X);
W and X are independently selected from hydrogen, substituted or un-substituted alkyl;
C is selected from hydrogen or —CO—N(Y)(Z);
Y and Z are independently selected from hydrogen, substituted or un-substituted alkyl.

This invention disclosed that R-bambuterol is a preferred selection from compound of formula I. Has the following structure as formula II

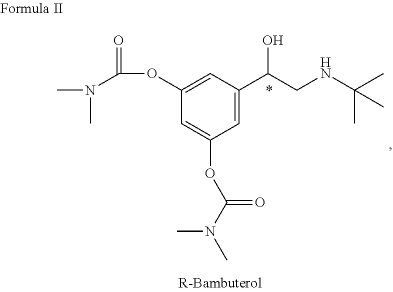

Wherein A is n-butyl, B is —CO—N(W)(X), W and X are methyl, C is —CO—N(Y)(Z), Y and Z are methyl according to formula I.

This invention disclosed that Mono-bambuterol is another preferred selection from formula I. Which has a structure as in formula III

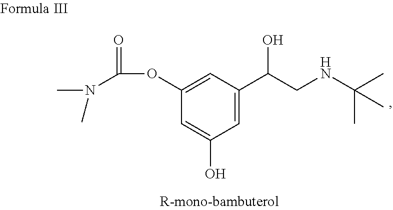 [SMILES: CN(C)C(=O)Oc1cc(O)cc(C(O)CNC(C)(C)C)c1 ; ","]

3-(2-(tert-butylamino)-1-hydroxyethyl)-5-hydroxyphenyl dimethylcarbamate bambuterol monocarbamate (MONO). Wherein A is n-butyl, B is —CO—N(W)(X), W and X are methyl, C is hydrogen according to formula I.

In one embodiment, we disclosed that mono-bambuterol, as did R-bambuterol, has similar effects in up-regulating LDL receptors and in enhancing intracellular LDL cholesterol clearances, as well as having synergistic effects and reduced toxicities when combined with either Atorvastatin or Simivastatin.

This invention disclosed that Ethylating bambuterol is also a preferred selection of formula I and has a structure as in formula IV.

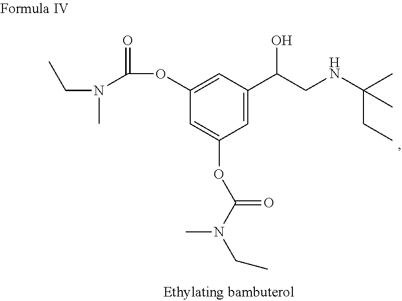 [SMILES: CCN(C)C(=O)Oc1cc(OC(=O)N(C)CC)cc(C(O)CNC(C)(C)CC)c1 ; ","]

5-(1-hydroxy-2-(tert-pentylamino)ethyl)-1,3-phenylene bis(ethyl(methyl) carbamate). Wherein A is tert-pentyl, B is —CO—N(W)(X), W is methyl, X is ethyl, C is —CO—N(Y)(Z), Y is methyl, Z is ethyl according to formula I.

In one embodiment, we disclosed that Ethylating bambuterol has similar effects as R-bambuterol, in up regulating LDL receptors and in enhancing intracellular LDL cholesterol clearances as well as having synergistic effects and reduced statins toxicities when combined with either Atorvastatin or Simivastatin.

This invention disclosed that (±)-carbamate-β-phenylethanolamine analogues upregulate LDL receptors, enhance the clearance of intracellular LDL cholesterol, and have synergistic effects on LDL clearance when combined with statins and other lipid lowering agents, in addition, combination of (±)-carbamate-β-phenylethanolamine analogues and statins can protect the cells against the toxicities induced by statins, therefore, can reduce the adverse effects of statins. All these disclosed findings by this invention have never been reported in prior art.

Furthermore, this invention overcomes a widely-accepted prejudice that R-bambuterol and its analogues play its lipid lowering roles via its parent drug terbutaline. This cannot be anticipated by a person in art.

Statin therapy in some cases may not effective enough to achieve the clinic required decrease of LDL cholesterol in patients. This is partially due to dose-limiting adverse effects, and also due to the efficacy of statin itself. Furthermore, there are about 30% patients do not response to statin therapy. Therefore, (±)-carbamate-β-phenylethanolamine analogues, disclosed in this invention provided new methods which can significantly improve the efficacy and effectiveness of current statin therapy, in addition, also provided a best alternatives for patients to whom statins are not effective.

This invention further disclosed that (±)-carbamate-β-phenylethanolamine analogues are specifically acting on LDL receptors, intracellular LDL clearance, in particular the liver cells. These have not been reported in prior art.

This invention disclosed methods of using R-bambuterol or carbamate-β-phenylethanolamine analogues as described in Formula I as combination therapy with statin or other lipid lowering agents such as Ezetimibe, Gemfibrozil, Fenofibrate acid (fibrates), Nicotinic acid, Cholestyramine or colestipol (resins), Cholesterol ester transfer protein (CETP) inhibitors, PKSC9 inhibitors such as Evolocumabor and Alirocumab, Bococizumab and Inclisiran for synergistic effects and for reduced adverse effects or toxicity to a patient in need.

These combination therapies is not just additive effects, instead, they provide a synergistic effects in efficacy and significantly reduce adverse effects. Therefore, these combination therapies disclosed in this invention provide a better therapeutic index for using statins and other lipid lowering agents to patients in need.

These new methods of combined use of (±)-carbamate-β-phenylethanolamine analogues or R-bambuterol with statins or other lipid lowering agents are novel and cannot be anticipated by a person in art.

This invention provides that the active enantiomers of compounds in formula I have better effects in up-regulating LDL receptor expression and binding and facilitating up-taking of LDL cholesterol and enhancing the clearance of intracellular LDL cholesterol. The active enantiomers of compounds in formula I also have better synergistic lipid lowering effects when used in combining with statins. At the same time, the active enantiomers of compounds in formula I have better protective effects against the toxicities of statins when used in combination.

In an embodiment in this invention provides an novel pharmaceutical composition comprising effective amount of R-bambuterol or a compound from formula I or their salts and Atorvastatin or one of the statins as a combined therapeutic preparation for used in combination therapy for simultaneous, sequential or separate administration by oral, inhale, injection, topically, rectal or vaginal administration to patients in need. The dosage forms are solid form, solutions, injectable form, ointment, soft capsule and suppository.

The ratio of the amount of active ingredient used in the combination therapy of statins and compounds from formula I may be adjusted depending on the therapeutic objectives of the use of the active agents and the age and condition of the patients. In the combination, there should have at least one statin in the amount of 1%-99% and at least one compound from formula I in the amount of 1%-99%.

The pharmaceutical acceptable salts of compound from formula I or R-bambuterol according to the invention include those formed with conventional pharmaceutical acceptable inorganic or organic acids for example: hydrochloride, hydrobromide, sulphate, hydrogen sulphate, dihydrogen phosphate, methanesulphonate, bromide, methyl sulphate, acetate, oxalate, maleate, fumarate, succinate, 2-naphthalene-sulphonate, glyconate, gluconate, citrate, tartaric, lactic, pyruvic isethionate, benzenesulphonate or para-toluenesulphonate.

### EXAMPLES

### Example 1

R-bambuterol increases expression of LDL receptors and facilitates up-taking of LDL-Cholesterol

Test Method:

Mouse liver AML12 cells were seeded in 24 well plates in DMEM/F12 and supplemented with 10% heat-inactivated fetal bovine serum. Cells were incubated with florescence labeled LDL to study the up-taking and metabolite of LDL cholesterol by the cells. LDL specific antibody were used to study LDL receptors expressed by the cells. Cells were divided into different groups and loaded with R-bambuterol (R-BM, Atorvastatin (Statin) or R-BM+Statin separately including: control, LDL, LDL+statin10 μM, LDL+R-BM 10 μM or 20 μM, LDL+R-BM10 μM+Statin10 μM LDL+R-BM20 μM+Statin20 μM groups. After 24 hours of treatments, the cells were washed and fixed with paraformaldehyde. Cells were then incubated with anti-LDLR primary antibody with 1:200 dilution at room temperature for 4 hours. After washed, the second antibody Alexa Fluor 488 with 1:200 dilutions was added and incubated at room temperature for 2 hours. The cells were washed again before examined under fluorescent microscope. The fluorescence anti-body bound LDL receptors in green and either bound or internalized fluoresces labeled LDL in red were examined separately or after merging. Fluorescent microscope equipped with a digital camera (Axio Observer 7). Computer software was used to quantify the intensity of fluorescence, which is correlated with the amount of LDL and LDL receptors bounded to antibodies.

### Test Results:

1) there are significantly more bound and intracellular fluorescence labeled LDL in groups treated with both 10 μM and 20 μM R-bambuterol in comparison of either control and LDL, LDL+Statin10 μM and LDL+statin20 μM. There is more bound and intracellular labeled LDL in R-bambuterol 20 μM than R-bambuterol 10 μM. This results indicate that R-bambuterol enhanced the transporting of LDL cholesterol into hepatic cells in a dose depend fashion. Hepatic cells is the major metabolic pathway for LDL cholesterol. Therefore, R-bambuterol facilitates the clearance of LDL from extracellular spaces or blood.

2), The bound and intracellular fluorescence labeled LDL in Atorvastatin treated group is more than LDL alone group but less than R-bambuterol treated groups. This indicates that Atorvastatin can also facilitate the LDL transport into cell.

3), The bound and intracellular fluorescence labeled LDL in group treated with combination of Statin and R-bambuterol are significantly more than either R-bambuterol or statin along treated groups. This results indicate a synergistic effects of R-bambuterol and Statin in LDL transporting into cells.

4), By quantification of the fluorescence intensity in randomly selected areas in the fields under microscope, the amount of LDL receptors in each group can be quantified as list in table below. LDL receptors were significantly unregulated with LDL loaded media but there is further increases of LDL receptors in R-bambuterol treated groups. The increases in LDL receptors expression in R-bambuterol treated cells was twice as much as seen in statin treated groups. These results indicate that R-bambuterol can up-regulate the expression of LDL receptors and it is more potent than statins in this regard.

However, the effects of statin were greatly enhanced when combined with R-bambuterol in comparison of statin alone.

TABLE 1
Up-regulating of LDL receptors by R-BM and Statin
in Hepatic cells (Intensity of fluoresces)

| Treatment (μM) | MEAN % | SD |
|---|---|---|
| control | 100.0 | 2.8 |
| R-BM 10 | 202.9 | 3.7 |
| R-BM 20 | 337.2 | 4.9 |
| Statin 10 | 159.1 | 5.4 |
| Statin 20 | 140.2 | 7.1 |
| R-BM20 + Statin20 | 226.5 | 7.3 |

### Example 2

R-bambuterol enhances the clearance or turnover LDL cholesterol and has synergistic effects with statin.

Human liver HepG2 cells were seeded in six well plates in DMEM/F12 supplemented with 10% heat-inactivated fetal bovine serum. Cells were then treated with different doses of R-bambuterol (R-BM) in the present of LDL (40 μg/ml). After 24 hours of incubation, total intracellular and extracellular cholesterol content were determined using Total Cholesterol Assay Kit E105 (Applygen Technologies Inc., Beijing, China) according to the manufacturer's protocol.

1) Enhancement of Clearance of Intracellular LDL Cholesterol

The intracellular cholesterol is similar in control and LDL groups. Neither statin of 10 μM nor R-bambuterol of 10 μM had any effects on the levels of intracellular cholesterol. However, in another experiment, mouse liver AML12 cells were treated with R-BM 10 μM and Statin 10 μM were used separately or in combination in the present of LDL (40 μg/ml), similar cell culture media and measurement methods of cholesterol were used.

### Test Results

There was a significant reduction of intracellular cholesterol when the same dose of R-bambuterol and Atorvastatin were added in combining. These indicate synergistic effects on the clearance or turnover of intracellular LDL cholesterol by the combining treatment of R-bambuterol and Atorvastatin.

TABLE 2
Reduction of intercellular LDL cholesterol by R-bambuterol

| Treatment (μM) | MEAN % | SD |
|---|---|---|
| LDL Control | 100 | |
| LDL + RBM 5 | 94.07 | 18.3 |
| LDL + RBM 10 | 69.26 | 13.6 |
| LDL + RBM 20 | 28.54 | 3.5 |
| LDL + RBM 40 | 36.04 | 12.2 |
| LDL + RBM 80 | 21.02 | 10.7 |

### 2) Synergistic Effects of R-Bambuterol and Statin in Reduction of Cholesterol

Unlike in HepG2 cells, R-BM 10 μM induce less reduction of intercellular cholesterol in Mouse liver AML12 cells. Similarly, Atorvastatin 10 μM also showed little effects on intercellular cholesterol. However, there was a significant more reduction in total cholesterol when cells were treated with a combination of both R-BM and Atorvastatin (Statin) at the same doses above. The reduction effects is more than an addition of both, therefore, there is a synergy for R-BM and Statin in the clearance of intracellular LDL cholesterol.

TABLE 3
Synergistic Effects of Combining R-bambuterol and Statin

| Treatment (μM) | MEAN % | SD |
|---|---|---|
| LDL Ctrl | 100.0 | 1.1 |
| LDL + Statin 10 | 95.3 | 7.8 |
| LDL + RBM 10 | 94.5 | 10.6 |
| LDL + Statin10 + RBM10 | 70.8 | 8.3 |

### Examples 3

Protective effects of R-bambuterol against the toxicities induced by statin.

### Test Method

Either HepG2 cell or Pulmonary artery smooth muscle cell (PASMC) were seeded evenly in 96-well plates. when cells grow about 50%, culture medium was replaced with FBS-free medium and Atorvastatin (Statin) or/and R-bambuterol (R-BM). Cells were cultivated for 24 hours at room temperature. The cells then replaced again with FBS-free medium containing 10% CCK-8 reagent and cultivated for 3 hours in the dark.

A Multi-function micro plate reader (TriStar2S LB942) was used to measure the cell viability.

Cells added no drugs (0 μM) were used as control. The viabilities from R-BM or statin or both were normalized as percent of control. For the combination of statin and R-BM treatment, R-BM was 20 μM for all groups except in control (OpM) in which no R-BM was added.

### Test Results

### 1) Hepatic Cells

R-bambuterol showed no toxicity to the viability of hepatic cells (HepG2) at all doses. However, Statin has significant toxic effects at initial dose of 5 μM, the viability of cells was inhibited by Statin in a dose-dependent fashion. The toxicity of statins was significantly reduced by combing with R-bambuterol, and completely abolished at lower dose of statin groups.

TABLE 4
Effects of R-bambuterol and Atorvastatin on the Viabilities
of Hepatic cells (Percent of control %)

| Treatment | 0 μM | 5 μM | 10 μM | 20 μM | 40 μM | 80 μM |
|---|---|---|---|---|---|---|
| Statin | 100 ± 9 | 81 ± 9 | 77 ± 8 | 76 ± 6 | 67 ± 1 | 51 ± 5 |
| R-BMB | 100 ± 7 | 103 ± 13 | 98 ± 12 | 105 ± 12 | 103 ± 7 | 101 ± 7 |
| Statin + R-BMB(20 μM) | 100 ± 5 | 98 ± 8 | 102 ± 4 | 93 ± 3 | 85 ± 5 | 63 ± 4 |

### 2) Pulmonary Artery Smooth Muscle Cell

R-bambuterol showed no toxicity to the viability of pulmonary artery smooth muscle cell (PASMC), PASMC seems more tolerable to Atorvastantin than HepG2 cells. However, there were also a dose-dependent inhibition of cell viability of PASMC when exposed to statin. Similarly, these toxicity of statins were significantly reduced by combining with R-bambuterol as in HepG2 cells.

TABLE 5
Effects of R-bambuterol and Atorvastatin on the
Viabilities of PASMC cells (Percent of control %)

| Treatment | 0 μM | 5 μM | 10 μM | 20 μM | 40 μM | 80 μM |
|---|---|---|---|---|---|---|
| Statin | 100 ± 6 | 92 ± 8 | 93 ± 2 | 88 ± 3 | 79 ± 2 | 68 ± 2 |
| Statin + | 100 ± 4 | 108 ± 2 | 100 ± 4 | 99 ± 6 | 84 ± 2 | 72 ± 3 |
| R-BM(20 μM) | | | | | | |

1. Methods of use compounds of (±)-carbamate-β-phenylethanolamine analogues described in formula I and their active enantiomers as well as their pharmaceutically suitable salts in a pharmaceutical composition in the manufacture of a medicament for up-regulating the activities of LDL receptors and for facilitating the up taking, internalization and clearance of intercellular LDL cholesterol in cells and for combination therapeutics with statins or other lipid lowering agents to achieve synergistic efficacy or reduce toxicity or adverse effects to a patient in need. 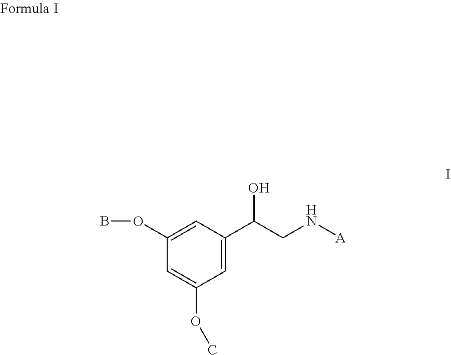
Wherein
A is selected from substituted or unsubstituted alkyl, substituted or unsubstituted alkenyl, substituted or unsubstituted aryl, substituted or unsubstituted cycloalkyl;
B is selected from hydrogen or —CO—N(W)(X);
W and X are independently selected from hydrogen, substituted or unsubstituted alkyl;
C is selected from hydrogen or —CO—N(Y)(Z);
Y and Z are independently selected from hydrogen, substituted or unsubstituted alkyl.

2. A method of claim 1, wherein said compounds is R-bambuterol presented in the structure formula II: 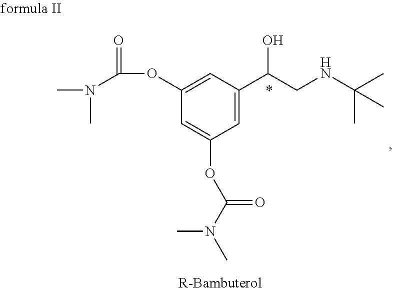
Wherein A is n-butyl, B is —CO—N(W)(X), W and X are methyl, C is —CO—N(Y)(Z), Y and Z are methyl according to Formula I.

3. A method of claim 1, wherein said compounds is R-mono-bambuterol presented in formula III: 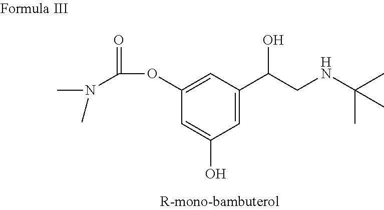 [SMILES: CN(C)C(=O)Oc1cc(O)cc(C(O)CNC(C)(C)C)c1]
3-(2-(tert-butylamino)-1-hydroxyethyl)-5-hydroxyphenyl dimethylcarbamate bambuterol mono-carbamate (MONO)
Wherein A is n-butyl, B is —CO—N(W)(X), W and X are methyl, C is hydrogen according to Formula I.

4. A method of claim 1, wherein the said compounds is Ethylating bambuterol Presented in the structure formula IV: 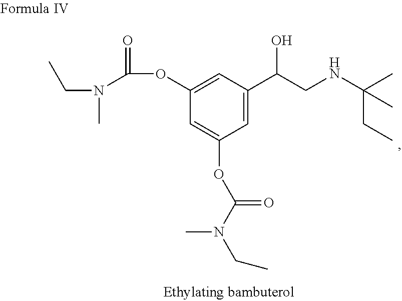 [SMILES: CCN(C)C(=O)Oc1cc(OC(=O)N(C)CC)cc(C(O)CNC(C)(C)CC)c1 ; ","]
5-(1-hydroxy-2-(tert-pentylamino)ethyl)-1,3-phenylene bis(ethyl(methyl) carbamate)
Wherein A is tert-pentyl, B is —CO—N(W)(X), W is methyl, X is ethyl, C is —CO—N(Y)(Z), Y is methyl, Z is ethyl according to Formula I.

5. A method of claim 1, wherein the said up-regulating activities comprise of increased expression of the receptors, increased binding of LDLC-LDL receptors and increased internalization of LDLC-LDL receptor complexes from cellular spaces into cells.

6. A method of claim 1, wherein the said clearance is reduced synthesis of LDL cholesterol.

7. A method of claim 1, wherein the said clearance is increased metabolize of LDL cholesterol.

8. A method of claim 1, wherein the said clearance is secretion of cholesterol in the form of bile acids.

9. A method of claim 1, wherein, the said cells is hepatic cells, muscle cells, smooth muscle cells, cardiac cells, endothelia cells, kidney cells, retina cells, neuronal cells, glia cells, macrophages and other cells which are capable of uptake or synthesis LDL cholesterol.

10. A method of claim 1, wherein, the said up-regulating activities of LDL receptors or clearance of LDL cholesterol involve an inhibition of butyric cholinesterase.

11. A method of claim 1, wherein, the said reduced toxicity involves an inhibition of butyric cholinesterase.

12. A method of claim 1, wherein, the said lipid lowering agents are statins.

13. A method of claim 12, wherein wherein the statin is selected from the group consisting of: atorvastatin, cerivastatin, fluvastatin, lovastatin, mevastatin, pitavastatin, pravastatin, rosuvastatin and simvastatin.

14. A method of claim 1, wherein, the lipid lowering agents are cholesterol absorption or transport inhibitors selected from the group consisting of Ezetimibe, Gemfibrozil, Fenofibrate acid (fibrates), Nicotinic acid, Cholestyramine or colestipol (resins), Cholesterol ester transfer protein (CETP) inhibitors.

15. A method of claim 1, wherein the said lipid lowering agents are PCSK 9 inhibitors selected from the group consisting of evolocumab, alirocumab, bococizumab and inclisiran.

16. A method of claim 1, wherein the combination therapy comprises of at least on of compounds of formula I and at least one lipid lowering agents according claim 1 for simultaneous, sequential or separate administration to a patient in need.

17. A method of claim 1, wherein the combination therapeutics comprise of at least one of compounds of formula I in the amount of 1-99% and at least one lipid lowering agents according claim I in the amount of 1-99%.

18. The method of claim 1, wherein the said pharmaceutical compositions are selected from the group consisting of tablets, capsules, granules, suppository, ointment, time-released dosage forms, skin patch, aqueous solutions and inhaled aerosol and for oral, topical, rector, vagina, parenteral injection, lung inhalation, nasal spray, or implant use.

19. The method of claim, wherein the pharmaceutical acceptable salts are selected from the groups of inorganic or organic acids consisting of hydrochloride, hydrobromide, sulphate, hydrogen sulphate, dihydrogen phosphate, methanesulphonate, bromide, methyl sulphate, acetate, oxalate, maleate, fumarate, succinate, 2-naphthalene-sulphonate, glyconate, gluconate, citrate, tartaric, lactic, pyruvic isethionate, benzenesulphonate or para-toluenesulphonate.
